(19) United States
(12) Patent Application Publication
(10) Pub. No.: US 2008/0056934 A1
(43) Pub. Date: Mar. 6, 2008

(54) DIFFUSIVE PLASMA AIR TREATMENT AND MATERIAL PROCESSING

(75) Inventor: Herman Yik Wai Tsui, Kowloon (HK)

Correspondence Address: BLAKELY SOKOLOFF TAYLOR & ZAFMAN, 1279 OAKMEAD PARKWAY, SUNNYVALE, CA, 94085-4040, US

(73) Assignee: Alphatech International Limited, Kowloon (HK)

(21) Appl. No.: 11/850,527

(22) Filed: Sep. 5, 2007

Related U.S. Application Data
(60) Provisional application No. 60/824,468, filed on Sep. 5, 2006.

(51) Int. Cl. A61L 9/16; A61L 9/00; B01J 19/00; B01J 19/12

(52) U.S. Cl. 422/2; 422/117; 422/121; 422/4

(57) ABSTRACT

The Diffusive Plasma is for effective treatment of contaminated air and material processing. Air is purified and disinfected by passing through the diffusive plasma device which includes a reactor or a plurality of reactors arranged in parallel or series and is energized by a high voltage alternating current power supply. The diffuser, being electrically isolated, provides extra nucleation sites to initiate discharges. It serves to improve the generation of uniform and consistent plasma and to reduce the variation of discharge properties among the reactors. The addition of a diffuser, thereby, enhances the overall effectiveness of decomposing chemicals and destroying microbes to achieve high air treatment and material processing performance. The diffuser can be made of suitable filtering materials to additionally serve as a filter. By incorporating suitable catalytic materials with the diffuser, the reactor becomes a catalytic plasma reactor wherein the plasma environment provides enhanced catalytic functions. Effective plasma power deposition may be obtained by controlling the amplitude, waveform period and shape of the voltage applied to the electrodes of the reactor and hence the operation of the reactors with plasma discharged of selected conditions for optimizing the treatment and processing efficiency while minimizing the generation of unwanted bi-product gases. The present invention also relates to a method for effective air treatment and material processing.

### CROSS-REFERENCE TO RELATED APPLICATIONS

This application claims priority from U.S. Provisional Patent Application No. 60/824,468, filed on Sep. 5, 2006, which is incorporated herein by reference in its entirety.

### TECHNICAL FIELD

The present invention relates generally to the application of plasma for air treatment and material processing and more particularly pertains to a plasma device for air purification and disinfection. The present invention also relates to a method for generating controllable and uniform plasma to improve the performance of air treatment and material processing.

### BACKGROUND

The following description of the background of the invention is provided to aid in understanding the invention, but is not admitted to describe or constitute prior art to the invention.

Plasma is referred to as the ‘4^{th} state of matter’, and is a partially ionized gas composed of freely moving ions, electrons, and neutral particles. While plasma is electrically neutral, it is electrically conductive. This property allows the injection of electrical energy into the space occupied by the plasma. Plasma is used today for a variety of commercial applications including air purification and disinfection (see for example, Ulrich Kogelschatz, Baldur Eliasson and Walter Egli, “From ozone generators to flat television screens: history and future potential of dielectric-barrier discharges”, Pure Appl. Chem., Vol. 71, No. 10, (1999) 1819). Depending on the operating conditions, plasma can consist of charged particles (electrons and ions), excited species, free radicals, ozone and UV photons, which are capable of decomposing chemical compounds and destroying microbes. The energy of the electrons can be utilized for exciting atoms and molecules, thereby initiating chemical reactions and/or emission of radiations. These emissions, particularly in the UV spectral region, can initiate photo-physical and photo-chemical process by breaking molecular bonds. The energetic electrons are able to induce the breakdown of some chemical bonds of the molecules, collide with the background molecules resulting in the breakdown of molecular chain, ionization and excitation, and generation of free atoms and radicals such as O, OH or HO₂. The radicals can attack hazardous organic molecules and are useful in decomposing pollutants in air. The disassociation of O₂ provides the required O to combine with O₂ to form ozone. The low energy electrons can attach to neutral atoms or molecules to form negative ions, which can enhance reactions in decomposing pollutants and destruction of microbes. The ability of plasma in destroying chemicals and deactivating microorganisms has been demonstrated.

Plasma can be created by electrical means in the form of gaseous discharges whereby a high voltage is applied to a set of electrodes, the anode and the cathode. When the applied voltage is sufficiently high and becomes greater than the breakdown voltage, arcs begin to develop across the electrodes. The threshold for electrical breakdown or arc formation follows the Paschen law, which relates the breakdown voltage to the gap size between the electrodes and the gas pressure.

Breakdown occurs when the applied voltage, or more precisely the local electric field, is sufficiently large for electrons to acquire enough energy to compensate the energy losses due to collisions, excitation and other energy loss processes. The breakdown process begins with the presence of some free or residual electrons accelerating towards the anode under the influence of the externally applied electric field. As they accelerate towards the anode, the streaming electrons collide with the gas atoms causing ionization directly by impact or indirectly through photo-ionization. An electron cloud begins to build up and propagates towards the anode together with an ionization or breakdown front ahead of the electron cloud, leaving an ion trail behind, resulting in a plasma channel with an electric dipole opposing the applied electric field. The formation of such a streamer or discharge filament, if unrestrained, leads to a rapid increase in charge density, fast growth of an avalanche, and the transformation of the streamer into an arc.

Traditionally, current limiting or quenching is achieved by placing an insulator or dielectric barrier to cover one or both electrodes in order to prevent the transformation of streamers or discharge filaments into major arcs and to establish a quasi-steady plasma state. The non-conductive property of the insulating or dielectric layer allows charge accumulation on the surface, which produces an opposite electric field to the applied electric field. In addition, the space charge built up next to insulating or dielectric layer adds to the electron repelling electric field. The opposing electric field cancels the applied electric field and prevents a filament from developing into a major arc and causes a discharge filament to extinguish. Therefore, the low charge mobility on the insulating or dielectric layer leads to self-arresting of the filaments and also limits their lateral extension, thereby allowing multiple filaments to form in close proximity to one another. Furthermore, when coalescence of multiple ionization fronts occurs, the filamentary discharge transforms to a diffuse a glow discharge that has spatially more uniform properties.

A number of other schemes are available to create a current limiting or quenching mechanism.
1. The applied voltage is carefully controlled to prevent transition into an arc;
2. Needle-like electrodes are used to create a space charge region around the smaller or sharper electrode (for example, as described in U.S. Pat. No. 6,042,637); and
3. Non-conductive materials are filled in the space between the electrodes as described in U.S. Pat. No. 4,954,320.

A typical plasma reactor for air treatment and material processing based on the above mentioned design principles generally suffer from unstable and variations in operation. One of the commonly encountered problems is the generation of quasi-steady filaments, i.e., filaments that reoccur persistently at the same location. While these filaments may not develop into arcs, their existence results in localized plasma generation and reduces the usefulness of the plasma for air treatment and material processing. For instance, effective air treatment requires harmful contaminants in air to have an adequate ‘residence time’ within the reactor device. Non-uniform plasma generation can reduce the treatment strength and thereby increase the required ‘residence time’ for treatment. The formation of these quasi-steady filaments can also lead to a higher generation of some undesirable by-product gases. Typical examples of bi-product gases are ozone and nitrogen dioxide (NO₂).

Therefore it is desirable to develop a method and a device that remedies at least some of the aforementioned problems.

### SUMMARY OF THE INVENTION

In view of the aforesaid disadvantages present in the prior art and based on the principles as mentioned above, the method and device of the present invention provides a process of generating plasma with more controllable and uniform properties so that plasma properties can be optimized to achieve better efficiency while minimizing the generation of unwanted bi-product gases.

The diffusive plasma device is a novel method and device to create plasma for air treatment and material processing. The diffusive plasma device generally comprises a reactor with a diffuser placed in the reaction chamber space between the two insulated electrodes powered by an alternating current power source. The reactor creates discharges directly to the air within the reactor chamber. The diffuser allows plasma properties to be modified for higher efficiency while minimizing the generation of unwanted bi-product gases. By incorporating suitable catalytic materials with the diffuser, the reactor becomes a catalytic plasma reactor wherein the plasma environment provides enhanced catalytic actions. With the use of suitable filtering materials, the diffuser can also act as a filter.

In a first preferred aspect, there is provided a system for treating air and processing materials, comprising:
at least one diffusive plasma reactor, each diffusive plasma reactor having insulated electrodes and a reaction chamber defined between the electrodes;
a diffuser located in the reaction chamber between the electrodes; and
a power supply for supplying high voltage alternating current to the electrodes;
wherein the electrodes generate plasma within the reaction chamber to treat air passing through the reaction chamber or process materials placed in the reaction chamber.

The diffuser may incorporate at least one predetermined material to enable the diffuser to also function as a filter or a catalyst.

The power supply may be adjustable to adjust the amplitude, waveform period and shape of the voltage applied to the electrodes so as to maximize plasma activity and minimize the generation of unwanted bi-product gases.

The at least one diffusive plasma reactor may be disposed in parallel arrangement with other diffusive plasma reactors in the system.

The system may further comprise a blower to drive air through the reaction chamber.

The system may further comprise an air filter to filter entering the reaction chamber.

Insulators of the electrodes may be in the form of a dielectric tube made of glass or plates.

Conductors of the electrodes may be made of conducting sheets, mesh or deposits.

The diffuser may be in the form of a sheet, a perforated sheet, a vertical sheet placed in between the electrodes, fan-folded between the electrodes, wire mesh, tangled string or fluff to loosely fill the space between the electrodes.

The diffuser may partially fill the reaction chamber between the electrodes such that the diffuser does not significantly affect the electrical properties of the diffusive plasma reactor and to maximum the availability of additional nucleation sites on electrically isolated surfaces of the diffuser.

The diffuser may be electrically isolated to allow accumulation of charge on its surfaces such that an opposite electric field to the applied electric field is generated to prevent the formation of localized quasi-steady filaments across the electrodes.

The voltage supplied may be in a range of 10 kilovolts to 50 kilovolts.

The waveform period may be in a range of 10⁻¹ ms to 10² ms.

The distance between a pair of electrodes may be in a range of 1 mm to about 20 mm.

In a second aspect, there is provided a method for air purification and disinfection, the method comprising:
providing at least one reactor, each reactor having insulated electrodes and a reaction chamber defined between the electrodes;
providing a diffuser in the reaction chamber between the electrodes;
supplying high voltage alternating current to the electrodes;
wherein plasma is generated within the reaction chamber by the electrodes for purifying and disinfecting air passing therethrough.

The method may further comprise adjusting the amplitude, waveform period and shape of the voltage applied to the electrodes to maximize plasma activity and minimize the generation of unwanted bi-product gases.

The electrodes are covered with insulating or dielectric material which provides a fundamental current limiting action. The diffuser can be made of electrically conductive or insulating materials. The diffuser is electrically isolated to provide extra nucleation sites to support the formation of discharge filaments. The material of the diffuser may only partially fill the space in the reaction chamber between the insulated electrodes such that it does not significantly affect the electrical properties of the reactor device. The diffusive plasma differs significantly from the reactive bed approach where the dielectric material is packed in the space between the electrodes to provide the fundamental current limiting action.

The device of the present invention has a high-voltage alternating current power source for controlling the amplitude, waveform period and shape of the voltage applied to the electrodes of the reactor and hence the operation of the reactor with plasma discharges of selected conditions. The high-voltage alternating current power source may be a high-voltage generator. The amplitude, waveform period and shape of the voltage applied to the electrodes may be adjusted according to the desired treatment strength and treatment time in the plurality of reactors.

The system generally comprises of a plurality of reactors arranged in parallel and/or in series allowing the configuration and size of each reactor be designed to result in a suitable treatment strength and time. The addition of a diffuser reduces the variation of discharge properties within each reactor and among the reactors, and thereby enhances the overall effectiveness of air treatment and material processing.

The insulated electrodes include insulators which may be in the form of dielectric tubes or plates. The diffuser may be made from conductive materials, though non-conductive or dielectric material is generally preferred. It may be in form of a sheet, a wire mesh, a tangled string or fluff.

The system may further include a blower unit for driving air through the reaction chambers. The system may further include an air filter.

It is an advantage of at least one embodiment of the present invention to produce more controllable plasma discharges for air treatment and material processing.

It is another advantage of at least one embodiment of the present invention to produce more uniform and consistent plasma properties.

It is another advantage of at least one embodiment of the present invention to allow more uniform and consistent plasma generation to achieve a high overall effectiveness of decomposing polluting chemicals and destroying microbes found in air.

It is another advantage of at least one embodiment of the present invention to maximize strength and effectiveness for treatment and processing.

It is a further advantage of at least one embodiment of the present invention to minimize the generation of unwanted bi-product gases.

It is a further advantage of at least one embodiment of the present invention to provide a method and device for air treatment which may be safe and reliable.

An even further advantage of at least one embodiment of the present invention is to provide a method and device for air treatment and material processing while minimizing the generation unwanted bi-product gases, thus overcoming the disadvantages of the prior art.

### BRIEF DESCRIPTION OF THE DRAWINGS

Specific embodiments of the invention will now be described by way of example with reference to the accompanying drawings wherein:

FIG. 1 illustrates the component assembly according to a preferred embodiment of the present invention;

FIG. 2a is a longitudinally-sectioned perspective view of a plasma device useful in the air treatment and material processing system according to a first embodiment of the present invention;

FIG. 2b is a perspective view of the plasma device of FIG. 2a;

FIG. 2c is a sectioned side view of the plasma device of FIG. 2a

FIG. 2d is an end view of the plasma device of FIG. 2a;

FIG. 3a is a longitudinally-sectioned perspective view of a plasma device useful in the air treatment and material processing system according to a second embodiment of the present invention;

FIG. 3b is a perspective view of the plasma device of FIG. 3a;

FIG. 3c is a sectioned side view of the plasma device of FIG. 3a;

FIG. 3d is an end view of the plasma device of FIG. 3a;

FIG. 4a is a longitudinally-sectioned perspective view of a plasma device useful in the air treatment and material processing system according to a third embodiment of the present invention;

FIG. 4b is a perspective view of the plasma device of FIG. 4a;

FIG. 4c is a sectioned side view of the plasma device of FIG. 4a;

FIG. 4d is an end view of the plasma device of FIG. 4a;

FIG. 5a is a longitudinally-sectioned perspective view of a plasma device useful in the air treatment and material processing system according to a fourth embodiment of the present invention;

FIG. 5b is a perspective view of the plasma device of FIG. 5a;

FIG. 5c is a sectioned side view of the plasma device of FIG. 5a;

FIG. 5d is an end view of the plasma device of FIG. 5a;

FIG. 6a is a perspective view of a reactor unit with a diffuser according to a first embodiment in planar geometry;

FIG. 6b is a perspective view of a reactor unit of FIG. 6a with a larger diffuser;

FIG. 6c is a sectioned side view of the reactor unit of FIG. 6a or FIG. 6b;

FIG. 6d is an end view of the reactor unit of FIG. 6a or FIG. 6b;

FIG. 7a is a perspective view of a reactor unit with a diffuser according to a second embodiment in planar geometry;

FIG. 7b is a perspective view of a reactor unit of FIG. 7a with a larger diffuser;

FIG. 7c is a sectioned side view of the reactor unit of FIG. 7a or FIG. 7b;

FIG. 7d is an end view of the reactor unit of FIG. 7a or FIG. 7b;

FIG. 8a is a perspective view of a reactor unit with a diffuser according to a third embodiment in planar geometry;

FIG. 8b is a perspective view of a reactor unit of FIG. 8a with a larger diffuser;

FIG. 8c is a sectioned side view of the reactor unit of FIG. 8a or FIG. 8b;

FIG. 8d is an end view of the reactor unit of FIG. 8a or FIG. 8b;

FIG. 9a is a perspective view of a reactor unit with a diffuser according to a fourth embodiment in planar geometry;

FIG. 9b is a perspective view of a reactor unit of FIG. 9a with a larger diffuser;

FIG. 9c is a sectioned side view of the reactor unit of FIG. 9a or FIG. 9b; and

FIG. 9d is an end view of the reactor unit of FIG. 9a or FIG. 9b;

### DETAILED DESCRIPTION OF THE INVENTION

Reference will now be made in detail to a preferred embodiment of the invention, examples of which are also provided in the following description. Exemplary embodiments of the invention are described in detail, although it will be apparent to those skilled in the relevant art that some features that are not particularly important to an understanding of the invention may not be shown for the sake of clarity.

Furthermore, it should be understood that the invention is not limited to the precise embodiments described below and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims.

In addition, improvements and modifications which may become apparent to persons of ordinary skill in the art after reading this disclosure, the drawings, and the appended claims are deemed within the spirit and scope of the present invention.

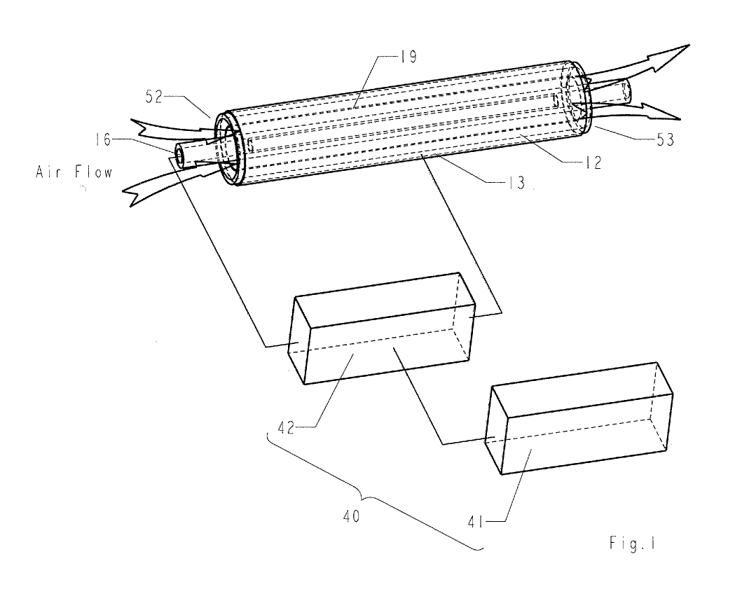

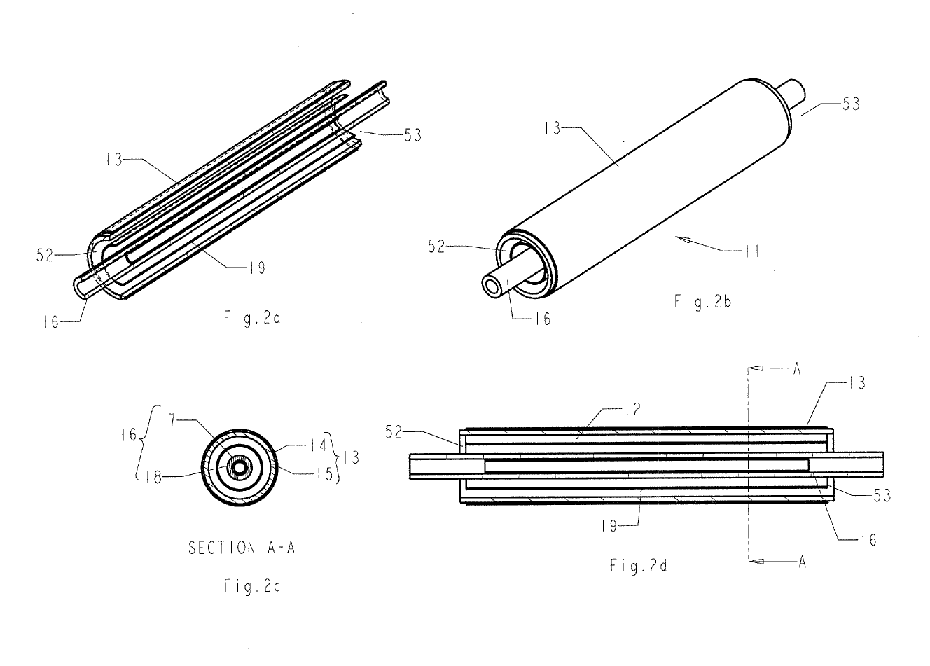

Referring now to the drawings, FIG. 1 generally shows system components of an air treatment system comprising the diffusive plasma reactor and its associated power supply and controller. The power supply and controller create and sustain discharges in the reactor with specific plasma parameters predetermined and controlled by the high-voltage alternating current power source. The set of FIGS. 2a, 2b, 2c and 2d show a preferred embodiment of a single reactor unit. As shown in this set of diagrams, each cylindrical reactor unit 11 includes an outer electrode 13 and an inner electrode 16 and both may be insulated from the annular space which forms the reaction chamber 12 where electrical discharges are excited to generate plasma. In the preferred embodiment, the insulators 15, 18 of the electrodes 13, 16 take the form of dielectric tube made of glass. They may also be in the form of plates or made from any insulating or dielectric material. The electrode conductors 14, 17 of the electrodes 13, 16 may be made of conducting sheets, mesh or deposits. A diffuser 19 is placed in the reaction chamber 12. The diffuser 19 may take many forms including but not limited to a sheet, a perforated sheet, wire mesh, tangled string or fluff as illustrated in the drawings of FIG. 2 through FIG. 5. (In these drawings, the equivalent components are labeled with the same last two digits, for example, the diffuser is labeled 19, 119, 219, 319 in FIG. 2 to FIG. 5 respectively.)

Electrical discharges are created in the reaction chamber 12 to generate plasma for air treatment. By circulating air through the plasma-filled reaction chamber 12, the pollutant particles and microbes in the air may be destroyed.

The diffuser 19 provides additional nucleation sites to support the formation of discharge filaments. To better perform this function, the diffuser 19 is electrically isolated. Although it can be made of conductive materials, a diffuser 19 made of non-conductive materials is better at producing consistent and uniform plasma. The diffuser 19 only partially fills the reaction chamber 12 between the insulated electrodes 13, 16 such that the diffuser 19 does not significantly affect the electrical properties of the reactor unit 11. (For example, the diffuser 19 does not significantly alter the capacitance of the reaction chamber 12.)

The purpose and arrangement of the diffuser 19 is different from the reactive bed designs. In a reactive bed design, the dielectric materials are packed in the space between the electrodes to provide the fundamental current limiting action. In a diffusive plasma reactor, the diffuser 19 is not meant to provide the fundamental current limiting function which is already provided by the insulators on the electrodes 13, 16. The diffuser 19 provides additional nucleation sites on its surfaces to support the formation of discharge filaments and to modify the local electric field structure. The diffuser 19 is electrically isolated to allow charge accumulation on its surfaces to generate an opposite electric field to the applied electric field. This prevents the formation of localized quasi-steady filaments across the two electrodes. Consequently, the generation of plasma is relatively more consistent and evenly distributed within the reaction chamber 12. The avoidance of concentrated filament formation eliminates the generation of unwanted bi-product gases from these localized areas.

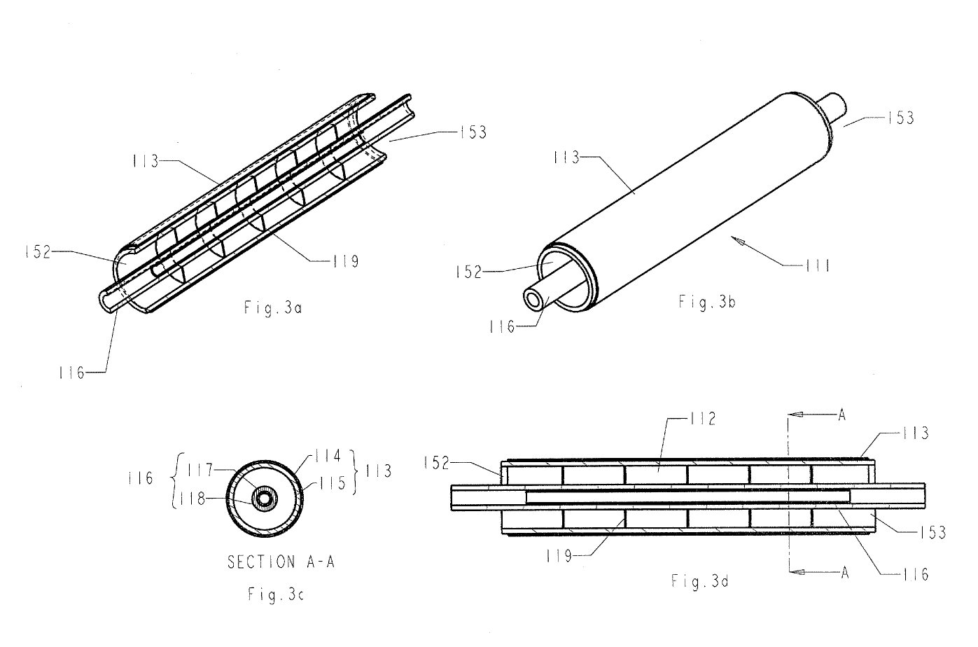

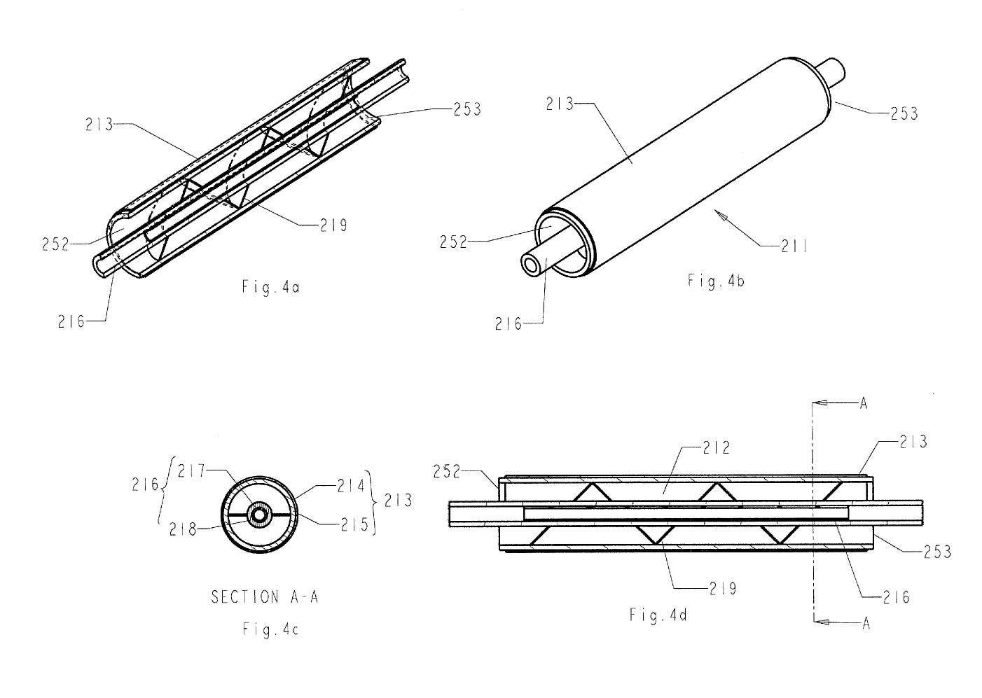

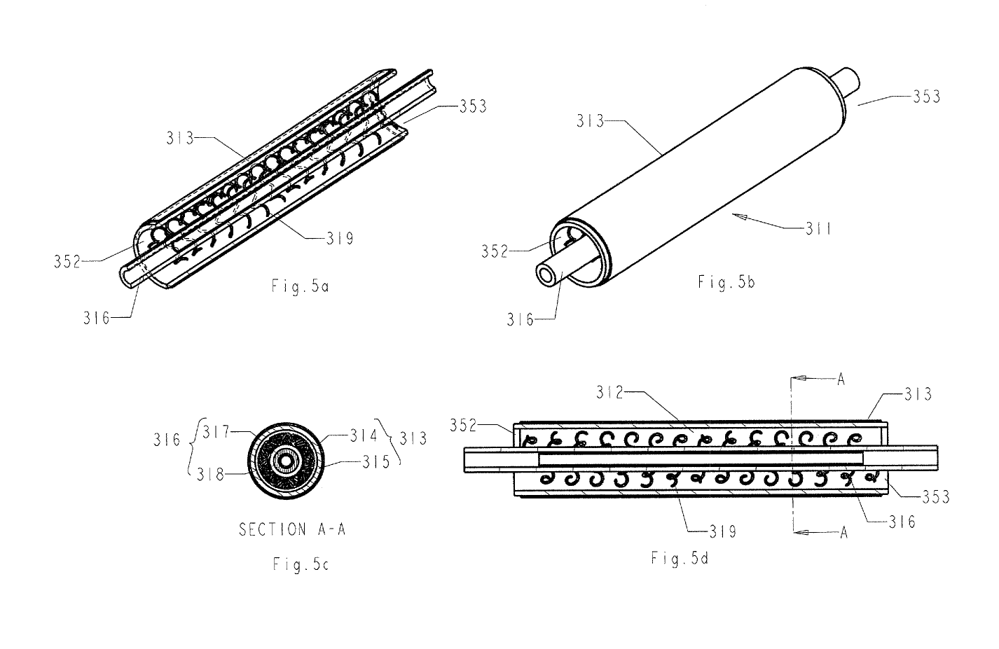

In a diffusive plasma reactor, the constituent materials of the diffuser 19 do not take up a significant portion of the volume within the reaction chamber 12 so that the availability of additional nucleation sites on the electrically isolated surfaces of the diffuser 19 is maximized. In contrast, a typical reactive bed design fills the space in the reaction chamber with dielectric packing materials. The physical arrangement of the diffuser 19 may be constructed differently. It can be in the form of a sheet of similar shape to the electrodes 13, 16 and be placed in the reaction chamber space between the electrodes 13, 16 (as illustrated in FIG. 2). The sheet can be perforated and even takes the form similar to a wire mesh. The diffuser 19 can also be arranged in the form of vertical sheets placed in between the electrodes 13, 16 (as illustrated in FIG. 3) or in a fan-folded manner fitted in between the electrodes 13, 16 (as illustrated in FIG. 4). The diffuser 319 can also be constructed like a tangled string or fluff that loosely fills up the space between the electrodes (as illustrated in FIG. 5).

By circulating air through the plasma-filled reaction chamber 12 incorporating the diffuser 19, the pollutant particles and microbes in the air are destroyed. The diffuser 19 may be constructed with suitable filtering materials to serve also serve as a filter. By incorporating suitable catalytic material with the diffuser 19, the reactor becomes a catalytic plasma reactor 11 wherein the plasma environment provides enhanced catalytic functions.

As illustrated in the schematic diagram FIG. 1, the electrodes 13, 16 may be connected to a high-voltage alternating current power supply 40 having an electronic control unit 41 and a high-voltage generator 42. The power supply 40 can provide sufficient voltage to cause breakdown and to generate plasma in the annular space of reaction chamber 12. The voltage applied to the electrodes 13, 16 may be controlled within a range of 10 kilovolts to 50 kilovolts. The waveform period may be controlled within a range of 10⁻¹ ms to 10² ms. The distance between a pair of insulated electrodes 13, 16 may be in the range of about 1 mm to about 20 mm.

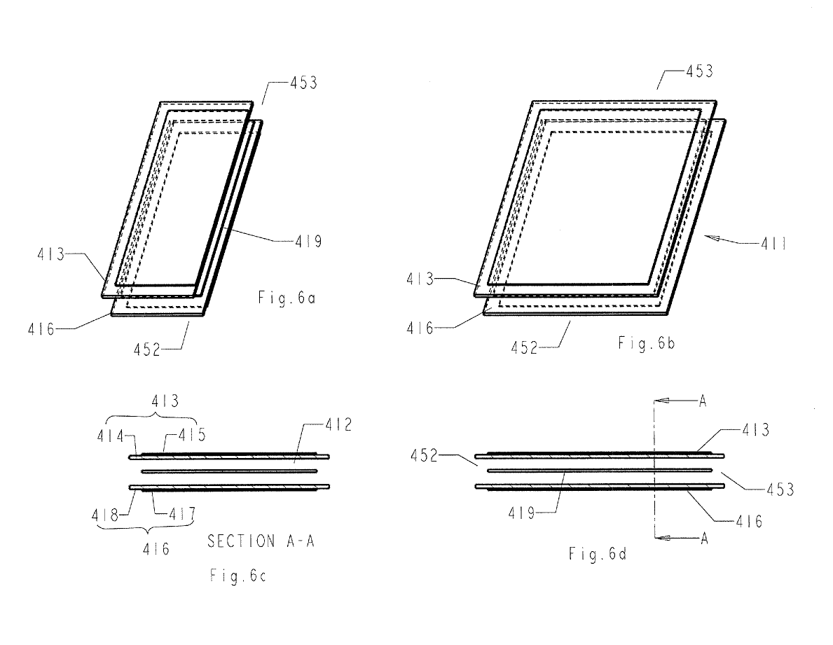

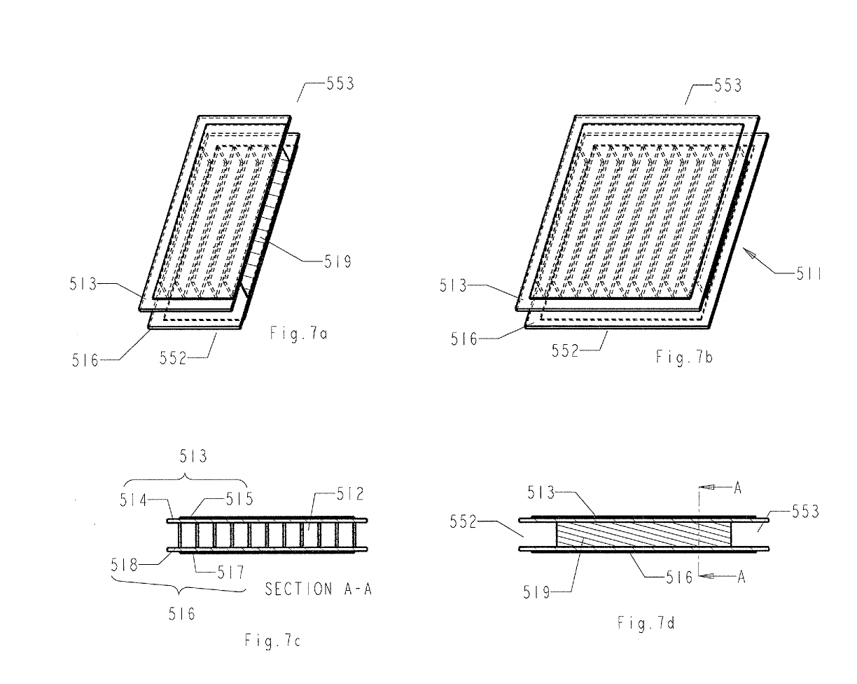

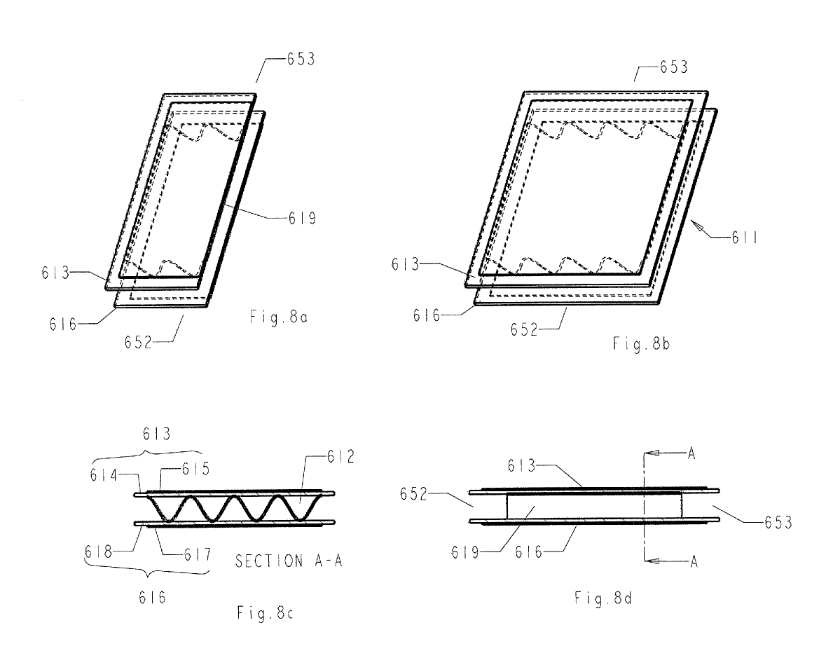

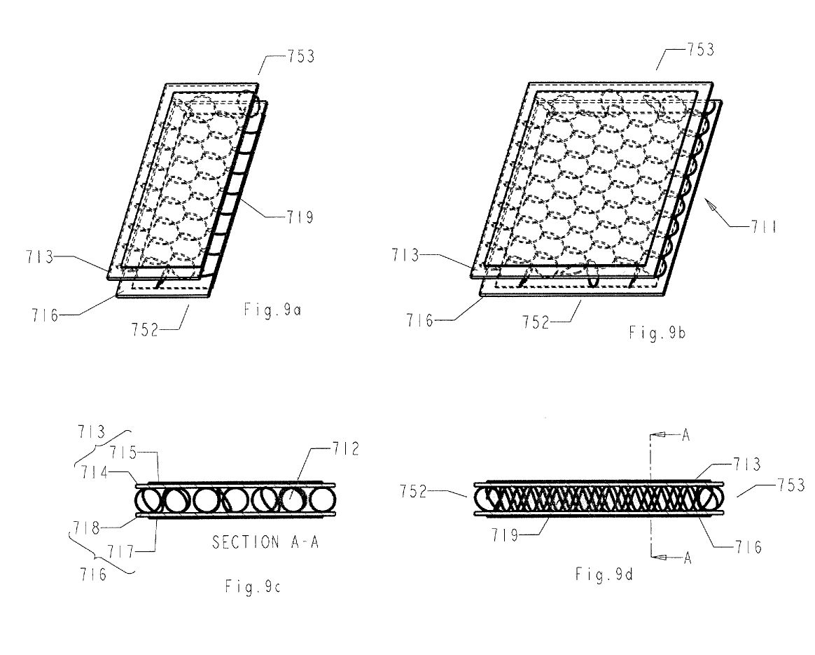

The device may be embodied, practiced and carried out in various ways. The drawings in FIGS. 6 to 9 show some alternative embodiments of the reactor unit 11 in planar geometry. Referring to FIG. 6, in one alternative embodiment, each reactor unit 411 consists of two insulated electrodes 413, 416 and a diffuser 419 placed in the reaction chamber 412 in between the electrodes 413, 416. In the illustrated embodiment, the insulators 415, 418 may take the form of glass or ceramic plate. The electrode conductors 414, 417 may be made of conducting sheets, mesh or deposits. The diffuser 419 may be constructed into many forms as illustrated in the drawings FIGS. 6 to 9. (In these drawings, the equivalent components are labeled with the same last two digits, for example, the diffuser is labeled 419, 519, 619 and 719 in FIG. 6 to FIG. 9 respectively.) The diffuser 419 in FIG. 6 is in the form of a sheet of similar shape to the electrodes 413, 416 and be placed in the space in the reaction chamber between the electrodes 413, 416. The sheet can be perforated and even takes the form similar to a wire mesh. The diffuser 519 can also be arranged in the form of vertical sheets placed in between the electrodes 513, 516 (as illustrated in FIG. 7) or in a fan-folded manner fitted in between the electrodes 613, 616 (as illustrated in FIG. 8). The diffuser 719 can also be constructed as tangled string or fluff that loosely fills up the space between the electrodes 713, 716 (as illustrated in FIG. 9).

It is to be understood that the phraseology and terminology employed herein are for the purpose of description and should not be regarded as limiting. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to falling within the scope of the invention.

What is claimed:

1. A system for treating air and processing materials, comprising:
at least one diffusive plasma reactor, each diffusive plasma reactor having insulated electrodes and a reaction chamber defined between the electrodes;
a diffuser located in the reaction chamber between the electrodes; and
a power supply for supplying high voltage alternating current to the electrodes;
wherein the electrodes generate plasma within the reaction chamber to treat air passing through the reaction chamber or process materials placed in the reaction chamber.

2. The system according to claim 1, wherein the diffuser incorporates at least one predetermined material to enable the diffuser to also function as a filter or a catalyst.

3. The system according to claim 1, wherein the power supply is adjustable to adjust the amplitude, waveform period and shape of the voltage applied to the electrodes so as to maximize plasma activity and minimize the generation of unwanted bi-product gases.

4. The system according to claim 1, wherein the at least one diffusive plasma reactor is disposed in parallel arrangement with other diffusive plasma reactors in the system.

5. The system according to claim 1, further comprising a blower to drive air through the reaction chamber.

6. The system according to claim 5, further comprising an air filter to filter air entering the reaction chamber.

7. The system according to claim 1, wherein insulators of the electrodes are in the form of a dielectric tube made of glass or plates.

8. The system according to claim 1, wherein conductors of the electrodes are made of conducting sheets, mesh or deposits.

9. The system according to claim 1, wherein the diffuser is in the form of a sheet, a perforated sheet, a vertical sheet placed in between the electrodes, fan-folded between the electrodes, wire mesh, tangled string or fluff to loosely fill the space between the electrodes.

10. The system according to claim 1, wherein the diffuser partially fills the reaction chamber between the electrodes such that the diffuser does not significantly affect the electrical properties of the diffusive plasma reactor and to maximum the availability of additional nucleation sites on electrically isolated surfaces of the diffuser.

11. The system according to claim 1, wherein the diffuser is electrically isolated to allow accumulation of charge on its surfaces such that the an opposite electric field to the applied electric field is generated to prevent the formation of localized quasi-steady filaments across the electrodes.

12. The system according to claim 1, wherein the voltage supplied is in a range of 10 kilovolts to 50 kilovolts.

13. The system according to claim 3, wherein the waveform period is a range of 10⁻¹ ms to 10² ms.

14. The system according to claim 1, wherein the distance between a pair of electrodes is in a range of 1 mm to about 20 mm.

15. A method for air purification and disinfection, the method comprising:
providing at least one reactor, each reactor having insulated electrodes and a reaction chamber defined between the electrodes;
providing a diffuser in the reaction chamber between the electrodes;
supplying high voltage alternating current to the electrodes;
wherein plasma is generated within the reaction chamber by the electrodes for purifying and disinfecting air passing therethrough.

16. The method according to claim 15, further comprising adjusting the amplitude, waveform period and shape of the voltage applied to the electrodes to maximize plasma activity and minimize the generation of unwanted bi-product gases.
